(19) United States
(12) Patent Application Publication
(10) Pub. No.: US 2016/0367183 A1
(43) Pub. Date: Dec. 22, 2016

(54) ELECTRONIC APPARATUS AND PHYSICAL ACTIVITY ASSISTANCE METHOD

(71) Applicant: Seiko Epson Corporation, Tokyo (JP)

(72) Inventor: Eiji Miyasaka, Okaya-shi (JP)

(21) Appl. No.: 15/185,200

(22) Filed: Jun. 17, 2016

(30) Foreign Application Priority Data
Jun. 19, 2015 (JP) 2015-124091

(51) Int. Cl. A61B 5/00; A61B 5/11; A61B 5/0205; A61B 5/22

(52) U.S. Cl. CPC A61B 5/486; A61B 5/224; A61B 5/112; A61B 5/1112; A61B 5/7278; A61B 5/0205; A61B 5/1118; A61B 5/681; A61B 5/6803; A61B 2560/0257; A61B 2562/0219; A61B 5/024

(57) ABSTRACT

An electronic apparatus includes a processor that generates, on the basis of evaluation of physical strength of a user who is performing physical activity and evaluation of the user's physical condition, information on whether or not the user's physical activity needs to be reconsidered.

### CROSS-REFERENCE

This application claims priority to Japanese Patent Application No. 2015-124091, filed Jun. 19, 2015, the entirety of which is hereby incorporated by reference.

### BACKGROUND

1. Technical Field

The present invention relates to an electronic apparatus, a physical activity assistance method, and a physical activity assistance program.

2. Related Art

When a person who is climbing a mountain or participating a running or bicycle competition or any other sport event feels fatigue or ill, the person needs to decide to properly take a rest or in some case, change the route.

There has been a proposed system that senses changes in environmental information and biological information that serve as indices of the physical condition of a person in a daily life and outputs advice on health of the person that allows the person to take care of the physical condition immediately on the spot on the basis of the changes (see JP-A-2013-196041 and others).

Such advice on health is, however, not necessarily information useful for a user at sport. In particular, during mountain climbing, items that should be taken into consideration in addition to the health are believed to be present in some cases to avoid risk of a mountain climbing accident.

### SUMMARY

An advantage of some aspects of the invention is to provide an electronic apparatus, a physical activity assistance method, and a physical activity assistance program capable of generating information that can serve as a supplement when a user who is performing a physical activity reconsiders the physical activity.

The invention can be implemented in the form of the following aspects or application examples.

### Application Example 1

An electronic apparatus according to this application example includes a processor that generates, on the basis of evaluation of physical strength of a user who is performing physical activity and evaluation of the user's physical condition, information on whether or not the user's physical activity needs to be reconsidered.

The processor generates, on the basis of both the user's physical strength and physical condition, the information on whether or not the physical activity needs to be reconsidered to reflect both the user's physical strength and physical condition in the information. The user can therefore use the information as a supplement for making appropriate judgement during the physical activity.

### Application Example 2

In the electronic apparatus according to the application example, the processor may perform the physical strength evaluation on the basis of a temporal change in the user's physical activity.

The processor uses a temporal change in the user's physical activity performance. A decrease in the user's physical strength or any other parameter over time can therefore be reflected in a result of the physical strength evaluation.

### Application Example 3

In the electronic apparatus according to the application example, the processor may perform the physical strength evaluation on the basis of a temporal change in at least one of altitude of a point where the user is present, the number of user's steps, the user's speed, the user's step width, and barometric pressure at the point where the user is present.

The processor uses a temporal change in at least one of the altitude, the number of steps, the speed, and the step width in the physical strength evaluation. These items tend to change, for example, as the physical strength of the user who is performing the physical activity changes. A change in the user's physical strength can therefore be reflected in a result of the physical strength evaluation.

### Application Example 4

In the electronic apparatus according to the application example, the processor may perform the physical strength evaluation on the basis of an output from at least one of a position sensor, an acceleration sensor, and an atmospheric pressure sensor.

The processor can use the output from at least one of the position sensor, the acceleration sensor, and the atmospheric pressure sensor in the physical strength evaluation.

### Application Example 5

In the electronic apparatus according to the application example, the processor may perform the physical condition evaluation on the basis of a temporal change in the user's biological information.

The processor uses a temporal change in the user's biological information in the physical condition evaluation. For example, decline in the user's physical condition over time can therefore be reflected in a result of the physical condition evaluation.

### Application Example 6

In the electronic apparatus according to the application example, the processor may perform the physical condition evaluation on the basis of a temporal change in the user's heart rate.

The processor can reflect a temporal change in the heart rate in a result of the physical condition evaluation.

### Application Example 7

In the electronic apparatus according to the application example, the processor may generate the information representing that the physical activity does not need to be reconsidered when a result of the physical strength evaluation and a result of the physical condition evaluation are both OK.

The processor generates information representing that the physical activity does not need to be reconsidered when a result of the evaluation of the user's physical strength and a result of the evaluation of the user's physical condition are OK. The information can prompt the user to continue the physical activity.

### Application Example 8

In the electronic apparatus according to the application example, the processor may generate the information representing that the physical activity needs to be changed when, out of a result of the physical strength evaluation and a result of the physical condition evaluation, only the result of the physical condition evaluation is NG.

The processor generates information representing that the physical activity needs to be changed when a result of the evaluation of the user's physical strength is not NG but a result of the evaluation of the user's physical condition is NG. The information can prompt the user to change the physical activity.

### Application Example 9

In the electronic apparatus according to the application example, the processor may generate the information representing that the physical activity needs to be temporarily stopped when, out of a result of the physical strength evaluation and a result of the physical condition evaluation, only the result of the physical strength evaluation is NG.

The processor generates information representing that the physical activity needs to be temporarily stopped when a result of the evaluation of the user's physical strength is NG but a result of the evaluation of the user's physical condition is not NG. The information can prompt the user to temporarily stop the physical activity.

### Application Example 10

In the electronic apparatus according to the application example, the processor may generate the information representing that the physical activity needs to be discontinued when a result of the physical strength evaluation and a result of the physical condition evaluation are both NG.

The processor generates information representing that the physical activity needs to be discontinued when a result of the evaluation of the user's physical strength is NG and a result of the evaluation of the user's physical condition is also NG. The information can prompt the user to discontinue the physical activity.

### Application Example 11

In the electronic apparatus according to the application example, the processor may generate the information on whether or not a plurality of users' physical activity needs to be reconsidered on the basis of the evaluation of the plurality of users' physical strength and the evaluation of the plurality of users' physical condition.

The processor generates information on whether or not the physical activity needs to be reconsidered on the basis of both the plurality of users' physical strength and physical condition to reflect both the plurality of users' physical strength and physical condition in the information. The plurality of user can therefore use the information as a supplement for making appropriate judgement during the physical activity.

### Application Example 12

In the electronic apparatus according to the application example, the processor may generate the information representing that the physical activity does not need to be reconsidered when results of the evaluation of all the plurality of users' physical strength and results of the evaluation of all the plurality of users' physical condition are both OK.

The processor generates information representing that the physical activity does not need to be reconsidered when results of the evaluation of all the plurality of users' physical strength and results of the evaluation of all the plurality of users' physical condition are OK. The information can prompt the plurality of users to continue the physical activity.

### Application Example 13

In the electronic apparatus according to the application example, the processor may generate the information representing that the physical activity needs to be changed when the number of users whose results of the physical condition evaluation, out of results of the physical strength evaluation and results of the physical condition evaluation, are only NG is greater than or equal to a predetermined value.

The processor generates information representing that the physical activity needs to be changed when the number of users whose results of the physical strength evaluation are not NG but results of the physical condition evaluation are NG is greater than or equal to a predetermined value. Even in the case of the plurality of users' physical activity, the information can prompt the users to change the physical activity.

### Application Example 14

In the electronic apparatus according to the application example, the processor may generate the information representing that the physical activity needs to be temporarily stopped when the number of users whose results of the physical strength evaluation, out of results of the physical strength evaluation and results of the physical condition evaluation, are only NG is greater than or equal to a predetermined value.

The processor generates information representing that the physical activity needs to be temporarily stopped when the number of users whose results of the physical strength evaluation are NG but results of the physical condition evaluation are not NG is greater than or equal to a predetermined value. The information can prompt the users to temporarily stop the physical activity.

### Application Example 15

In the electronic apparatus according to the application example, the processor may generate the information representing that the physical activity needs to be discontinued when the number of users whose results of the physical strength evaluation and results of the physical condition evaluation are both NG is greater than or equal to a predetermined value.

The processor generates information representing that the physical activity needs to be discontinued when the number of users whose results of the physical strength evaluation and the physical condition evaluation are NG is greater than or equal to a predetermined value. The information can prompt the users to discontinue the physical activity.

### Application Example 16

In the electronic apparatus according to the application example, the processor may provide the user with the information.

The processor, for example, can provide information on a certain user to the user himself/herself or another user. The processor can therefore notify the user himself/herself or the other of the information.

### Application Example 17

The electronic apparatus according to the application example may be attachable to a predetermined site of the user.

The user can therefore use the electronic apparatus without holding the electronic apparatus by a hand.

### Application Example 18

In the electronic apparatus according to the application example, the predetermined site may be an arm or a wrist.

The user can therefore use the electronic apparatus as if it were, for example, a wristwatch.

### Application Example 19

A physical activity assistance method according to this application example includes generating, on the basis of evaluation of physical strength of a user who is performing physical activity and evaluation of the user's physical condition, information on whether or not the user's physical activity needs to be reconsidered.

The physical activity assistance method according to the application example generates, on the basis of both the user's physical strength and physical condition, the information on whether or not the physical activity needs to be reconsidered to reflect both the user's physical strength and physical condition in the information. The user can therefore use the information as a supplement for making appropriate judgement during the physical activity.

### Application Example 20

A physical activity assistance program according to this application example causes a computer to generate, on the basis of evaluation of physical strength of a user who is performing physical activity and evaluation of the user's physical condition, information on whether or not the user's physical activity needs to be reconsidered.

The computer generates, on the basis of both the user's physical strength and physical condition, the information on whether or not the physical activity needs to be reconsidered to reflect both the user's physical strength and physical condition in the information. The user can therefore use the information as a supplement for making appropriate judgement during the physical activity.

### BRIEF DESCRIPTION OF THE DRAWINGS

The invention will be described with reference to the accompanying drawings, wherein like numbers reference like elements.

FIG. 1 describes an overview of an electronic apparatus according to a first embodiment.

FIG. 2 is a functional block diagram for describing the configuration of the electronic apparatus.

FIG. 3 describes an example of a route.

FIG. 4 describes data on a route.

FIGS. 5A to 5C are flowcharts for describing physical strength evaluation, physical condition evaluation, and route search.

FIG. 6 shows the flow of data in the physical strength evaluation, the physical condition evaluation, and the route search.

FIG. 7 is a flowchart for describing individual-assistance-related action of a processor.

FIG. 8 describes a system including a plurality of electronic apparatus.

FIG. 9 describes the flow of data in the system.

FIG. 10 is a flowchart for describing party-assistance-related action of the electronic apparatus (master module).

FIGS. 11A and 11B show other examples of the exterior appearance of the electronic apparatus.

### DESCRIPTION OF EXEMPLARY EMBODIMENTS

A preferable embodiment of the invention will be described below in detail with reference to the drawings. The embodiment described below is not intended to unduly limit the contents of the invention set forth in the appended claims. Further, all configurations described below are not necessarily essential configuration requirements of the invention.

### 1. Embodiment of Electronic Apparatus

### 1-1. Overview of Electronic Apparatus

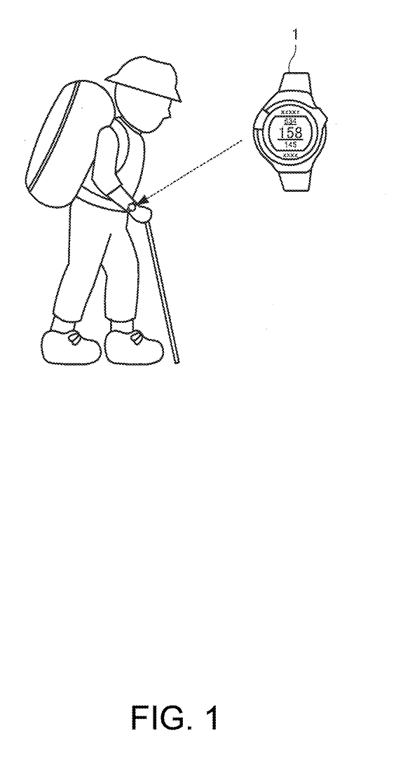

FIG. 1 describes an overview of an electronic apparatus according to a first embodiment.

An electronic apparatus (as an example, sport activity recording apparatus in the present embodiment) 1 according to the present embodiment is, for example, a mobile information apparatus attached to part of a user's body in an outdoor activity, such as mountain climbing, as shown in FIG. 1. The body part to which the electronic apparatus 1 is attached is, for example, a site in any position from the elbow to the hand (forearm) so that the electronic apparatus 1 is visible to the user whenever necessary. In the example shown in FIG. 1, the electronic apparatus 1 is configured as a wrist-worn-type (wristwatch-type) mobile information apparatus (outdoor watch), and the body part to which the electronic apparatus 1 is attached is the wrist.

The electronic apparatus 1 is provided with a positioning function, a compass function, an atmospheric pressure detection function, a pulse detection function, and a variety of other sensing functions as well as a clock function. The following description will be made on the assumption that the electronic apparatus 1 is used in mountain climbing (example of physical activity). The electronic apparatus 1 uses the sensing functions to assist the user who is climbing a mountain. The assistance is a process of monitoring the user's physical strength and physical condition, generating information on whether or not the mountain climbing needs to be reconsidered (assistance information for prompting user to continue mountain climbing, change plan of mountain climbing, temporarily stop mountain climbing, or discontinue mountain climbing) at appropriate timing, and providing the user with the information. The assistance will be described later in detail.

### 1-2. Configuration of Electronic Apparatus

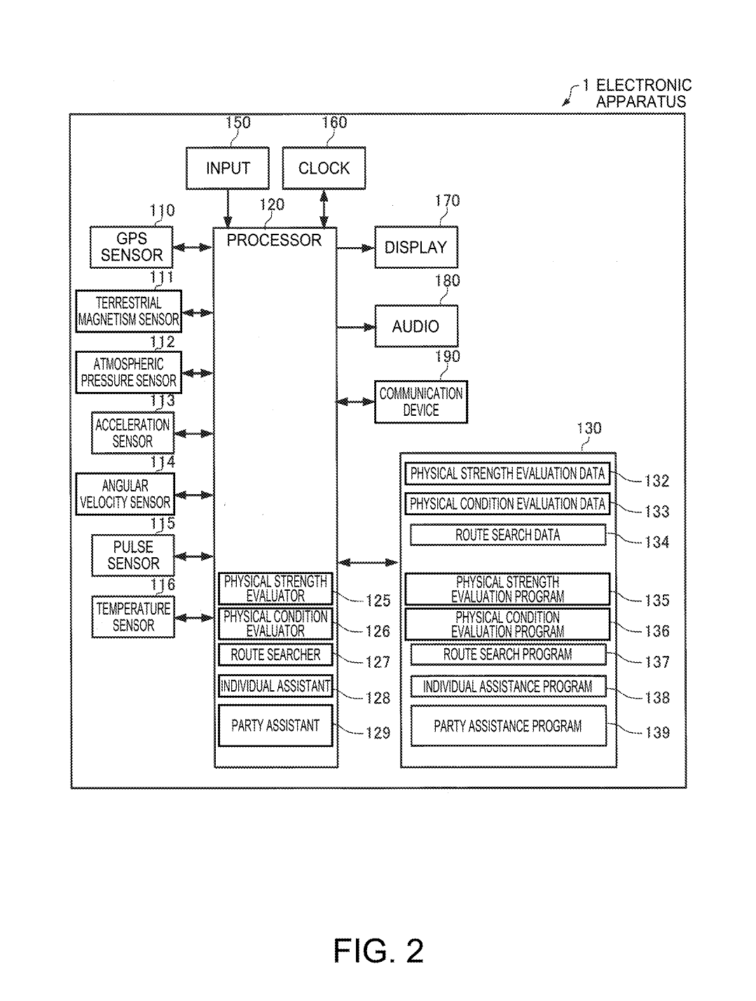

FIG. 2 is a functional block diagram for describing the configuration of the electronic apparatus.

The electronic apparatus 1 includes a GPS sensor 110, a terrestrial magnetism sensor 111, an atmospheric pressure sensor 112, an acceleration sensor 113, an angular velocity sensor 114, a pulse sensor 115, a temperature sensor 116, a processor 120, a memory 130, an input 150, a clock 160, a display 170, an audio 180, a communication device 190, and other components, as shown in FIG. 2. In the configuration of the electronic apparatus 1, however, part of the constituent elements described above maybe omitted or changed, or another constituent element (humidity sensor, ultraviolet ray sensor, for example) may be added.

The GPS sensor 110 is a sensor that produces positioning data (data on latitude, longitude, altitude, velocity vector, and other quantities) representing the position and other factors of the electronic apparatus 1 and outputs the positioning data to the processor 120, and the GPS sensor 110 includes, for example, a GPS (global positioning system) receiver and other components. The GPS sensor 110 receives an externally incoming electromagnetic wave that belongs to a predetermined frequency band via a GPS antenna that is not shown, extracts a GPS signal transmitted from a GPS satellite, and produces the positioning data on the basis of the GPS signal.

The terrestrial magnetism sensor 111 is a sensor that detects a terrestrial magnetism vector representing the terrestrial magnetic field direction viewed from the electronic apparatus 1 and produces, for example, terrestrial magnetism data representing magnetic flux densities in three axial directions perpendicular to each other. The terrestrial magnetism sensor 111 is formed, for example, of an MR (magnet resistive) element, an MI (magnet impedance) element, or a Hall element.

The atmospheric pressure sensor 112 is a sensor that detects the atmospheric pressure (barometric pressure) around the electronic apparatus 1 and has, for example, a pressure sensitive element operating on the basis of a method using a change in the resonance frequency of a vibrating piece (vibration method). The pressure sensitive element is a piezoelectric vibrator made, for example, of quartz, lithium niobate, lithium tantalate, or any other piezoelectric material and is, for example, a tuning-fork-type vibrator, a dual-tuning-fork-type vibrator, an AT vibrator (thickness shear vibrator), or an SAW resonator. The output from the atmospheric pressure sensor 112 may be used to correct position information contained in the positioning data.

The acceleration sensor 113 is an inertia sensor that detects acceleration in each of three axial directions that intersect each other (ideally perpendicular to each other) and outputs a digital signal according to the magnitude and direction of the detected acceleration in each of the three axes (acceleration data). The output from the acceleration sensor 113 may be used to correct the position information contained in the positioning data.

The angular velocity sensor 114 is an inertia sensor that detects angular velocity in each of the three axial directions that intersect each other (ideally perpendicular to each other) and outputs a digital signal according to the magnitude and direction of the measured angular velocity in each of the three axes (angular velocity data). The output from the angular velocity sensor 114 may be used to correct the position information contained in the positioning data.

The pulse sensor 115 is a sensor that produces a signal representing the user's pulse (heart rate) (example of biological information) and outputs the signal to the processor 120. The pulse sensor 115 includes, for example, an LED light source or any other light source that emits measurement light having an appropriate wavelength toward a subcutaneous blood vessel and a light receiving device that detects a change in the intensity of light produced at the blood vessel in response to the measurement light.

The temperature sensor 116 is a temperature sensitive element that outputs a signal according to the temperature around the electronic apparatus 1 (voltage according to temperature, for example). The temperature sensor 116 may instead be a sensor that outputs a digital signal according to the temperature.

The processor 120 is formed, for example, of an MPU (micro processing unit), a DSP (digital signal processor), an ASIC (application specific integrated circuit), and other components. The processor 120 carries out a variety of processes in accordance with a program stored in the memory 130 and a variety of commands inputted by the user via the input 150. The processes carried out by the processor 120 include data processing in which data produced by at least one of the GPS sensor 110, the terrestrial magnetism sensor 111, the atmospheric pressure sensor 112, the acceleration sensor 113, the angular velocity sensor 114, the pulse sensor 115, the temperature sensor 116, the clock 160, and other components are processed, display processing in which the display 170 is caused to display an image, audio processing in which the audio 180 is caused to output audio, and other types of processing.

The processor 120 operates in accordance with a physical strength evaluation program 135 stored in the memory 130 to function as a physical strength evaluator 125. The processor 120 further operates in accordance with a physical condition evaluation program 136 stored in the memory 130 to function as a physical condition evaluator 126. The processor 120 further operates in accordance with a route search program 137 stored in the memory 130 to function as a route searcher 127. The processor 120 further operates in accordance with an individual assistance program 138 (example of physical activity assistance program) stored in the memory 130 (this operation is an example of the physical activity assistance method) to function as an individual assistant 128. The processor 120 further operates in accordance with a party assistance program 139 (example of physical activity assistance program) stored in the memory 130 (this operation is an example of the physical activity assistance method) to function as a party assistant 129.

The memory 130 is formed, for example, of one or more IC memories and has a ROM that memorizes programs and other types of data and a RAM that serves as a work area used by the processor 120. The RAM includes a nonvolatile RAM, and the nonvolatile RAM provides a memory area where physical strength evaluation data 132, physical condition evaluation data 133, route search data 134, and other types of data are memorized. The programs memorized in the ROM include the physical strength evaluation program 135, the physical condition evaluation program 136, the route search program 137, the individual assistance program 138, the party assistance program 139, and other programs.

The input 150 is formed, for example, of buttons, keys, a microphone, a touch panel, an audio recognition function (using microphone that is not shown), and an action detection function (using acceleration sensor 113 and others), converts an instruction from the user into an appropriate signal, and transmits the signal to the processor 120.

The clock 160 is formed, for example, of a real-time clock (RTC) IC, produces time data, such as the year, month, date, hour, minute, and second, and transmits the time data to the processor 120.

The display 170 is formed, for example, of an LCD (liquid crystal display), an organic EL (electroluminescence) display, an EPD (electrophoretic display), or a tough-panel-type display and displays a variety of images in accordance with an instruction from the processor 120.

The audio 180 is formed, for example, of a loudspeaker, a buzzer, or a vibrator and produces a variety of type of audio (or vibration) in accordance with an instruction from the processor 120.

The communication device 190 performs a variety of types of control for establishing data communication between the electronic apparatus 1 and an external apparatus. The communication device 190 includes a transceiver that complies, for example, with Bluetooth (registered trademark) (including BTLE: Bluetooth Low Energy), Wi-Fi (registered trademark) (Wi-Fi: wireless fidelity), Zigbee (registered trademark), NFC (near field communication), ANT+ (registered trademark), or any other short-distance wireless communication standard.

### 1-3. Route Data

In the memory 130, data on a plurality of routes are registered in advance as the route search data 134. The data on a plurality of routes are registered in advance by the user in the electronic apparatus 1, for example, via a smartphone, a tablet PC, or any other information terminal (not shown). Data on a route is, for example, open to the public over the Internet or provided by a specific network server to the user over the Internet.

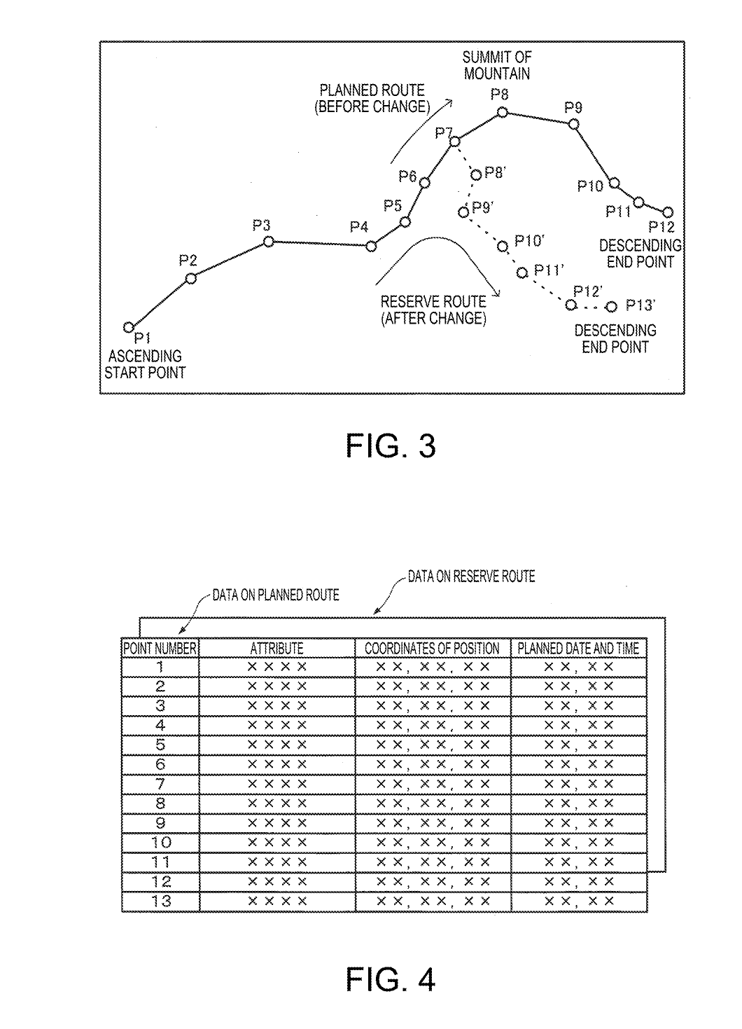

FIG. 3 describes an example of a route.

The route includes a planned route and a reserve route, as shown in FIG. 3. The planned route is a route along which the user plans to travel in the mountain climbing, and the reserve route is a route that can be selected when the user changes the route in the course of the mountain climbing (when the user seeks to quickly descend the mountain). FIG. 3 shows only one reserve route, but two or more reserve routes may be registered in the route search data 134.

The data on the route contains data on the coordinates (latitude, longitude, and altitude) of the positions of points P1, P2, P3, . . . from a start point to an end point of the route, as shown in FIG. 4. Each of the “points” used herein is a position on the route. The “points” may include points where a user plans to do some events (such as rest and lunch) (event points), may include a point where another route branches off, or may include any other point. The data on the route may contain data representing an attribute of each of the points, data on planned date and time when the user passes a point, and other types of data.

### 1-4. Physical Strength Evaluation

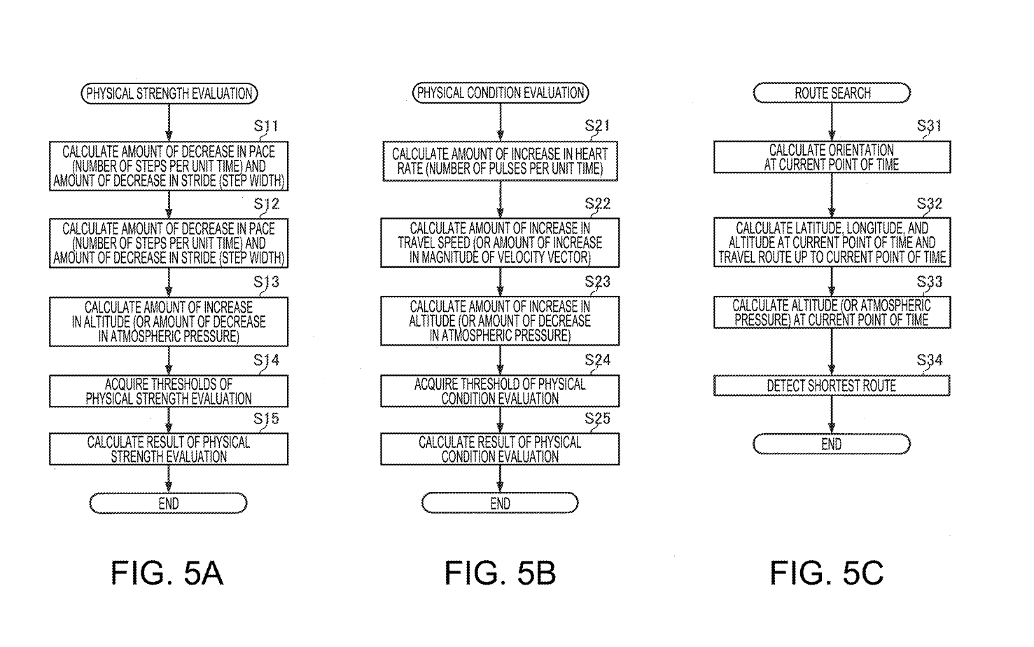

FIG. 5A is a flowchart for describing physical-strength-evaluation-related action of the processor 120. The physical strength evaluation is performed in accordance with the physical strength evaluation program 135. The timing at which the physical strength evaluation is performed is, for example, timing at which the physical strength needs to be evaluated in individual assistance, which will be described later. Each step in the physical strength evaluation will be sequentially described below.

Step S11: The processor 120 calculates the amount of decrease in the user's pace (number of steps per unit time) and the amount of decrease in the user's stride (step width) on the basis of a temporal change in the acceleration data (vertical component of acceleration data) outputted from the acceleration sensor 113.

The “amount of decrease in the pace” used herein is the amount of decrease in the current pace with respect to the pace a predetermined period before (for example, the amount of decrease with respect to the pace one hour before), and the “amount of decrease in the stride” used herein is the amount of decrease in the current stride with respect to the stride a predetermined period before (for example, the amount of decrease with respect to the stride one hour before).

Step S12: The processor 120 calculates a decrease in the user's travel speed on the basis of a temporal change in the position coordinates (or velocity vector) contained in the positioning data outputted from the GPS sensor 110.

The “amount of decrease in the travel speed” used herein is the amount of decrease in the current travel speed with respect to the travel speed a predetermined period before (for example, the amount of decrease with respect to the travel speed one hour before).

S13: The processor 120 calculates the amount of increase in the altitude of the point where the user is present (or the amount of decrease in the barometric pressure) on the basis of a temporal change in atmospheric pressure data outputted from the atmospheric pressure sensor 112.

The “amount of increase in the altitude” used herein is the amount of increase in the current altitude with respect to the altitude a predetermined period before (for example, the amount of increase with respect to the altitude one hour before), and the “amount of decrease in the barometric pressure” used herein is the amount of decrease in the current barometric pressure with respect to the barometric pressure a predetermined period before (for example, the amount of decrease with respect to the barometric pressure one hour before).

Step S14: The processor 120 refers to the physical strength evaluation data in accordance with the amount of increase in the altitude (or the amount of decrease in the barometric pressure) to acquire thresholds corresponding to the amount of increase in the altitude (or the amount of decrease in the barometric pressure).

The physical strength evaluation data stores thresholds for each amount of increase in the altitude (or for each amount of decrease in the barometric pressure), and the thresholds are formed of a pace threshold, astride threshold, and a travel speed threshold.

Step S15: The processor 120 compares the amount of decrease in the pace with the pace threshold, compares the amount of decrease in the stride with the stride threshold, and compares the amount of decrease in the travel speed with the travel speed threshold. The processor 120 then sets a result of the physical strength evaluation to be “NG” when the amounts of decrease in all the three parameters are greater than the thresholds and sets a result of the physical strength evaluation to be “OK” when the amount of decrease in at least one of the three parameters is smaller than or equal to the corresponding threshold.

The physical strength evaluation data stores in advance optimum thresholds for each amount of increase in the altitude (or the amount of decrease in the barometric pressure) with the thresholds related to the amount. Specifically, in the physical strength evaluation data, the greater the amount of increase in the altitude (or the amount of decrease in the barometric pressure), the greater the related threshold.

Therefore, for example, even though the route along which the user is traveling is flat, when the pace, the stride, and the travel speed significantly decrease, a result of the physical strength evaluation is set to be “NG,” and even though the route along which the user is traveling is a steep upslope, when the pace and the other parameters (pace, stride, and travel speed) only slightly decrease, a result of the physical strength evaluation is set to be “OK.”

In the physical strength evaluation described above, the order in which the steps described above are carried out can be changed as appropriate. For example, the points of time at which steps S11, S12, and S13 are carried out can be swapped with each other.

### 1-5. Physical Condition Evaluation

FIG. 5B is a flowchart for describing physical-condition-evaluation-related action of the processor 120. The physical condition evaluation is performed in accordance with the physical condition evaluation program 136. The timing at which the physical condition evaluation is performed is, for example, timing at which the physical condition needs to be evaluated in individual assistance, which will be described later. Each step in the physical condition evaluation will be sequentially described below.

Step S21: The processor 120 calculates the amount of increase in the user's heart rate (the number of pulses per unit time) on the basis of a temporal change in pulse data outputted from the pulse sensor 115.

The “amount of increase in the heart rate” used herein is the amount of increase in the current heart rate with respect to the heart rate a predetermined period before (for example, the amount of increase with respect to the heart rate one hour before).

Step S22: The processor 120 calculates the amount of increase in the user's travel speed on the basis of a temporal change in the position coordinates (or velocity vector) contained in the positioning data outputted from the GPS sensor 110.

The “amount of increase in the travel speed” used herein is the amount of increase in the current travel speed with respect to the travel speed a predetermined period before (for example, the amount of increase with respect to the travel speed one hour before).

Step S23: The processor 120 calculates the amount of increase in the altitude of the point where the user is present (or the amount of decrease in the barometric pressure) on the basis of a temporal change in the atmospheric pressure data outputted from the atmospheric pressure sensor 112.

The “amount of increase in the altitude” used herein is the amount of increase in the current altitude with respect to the altitude a predetermined period before (for example, the amount of increase with respect to the altitude one hour before), and the “amount of decrease in the barometric pressure” used herein is the amount of decrease in the current barometric pressure with respect to the barometric pressure a predetermined period before (for example, the amount of decrease with respect to the barometric pressure one hour before).

Step S24: The processor 120 refers to the physical condition evaluation data in accordance with the combination of the amount of increase in the travel speed and the amount of increase in the altitude (or the amount of decrease in the barometric pressure) to acquire a threshold corresponding to the combination.

The physical condition evaluation data stores thresholds for each set of values of the combination, and the threshold is a heart rate threshold.

Step S25: The processor 120 compares the amount of increase in the heart rate with the heart rate threshold. The processor 120 then sets a result of the physical condition evaluation to be “NG” when the amount of increase in the heart rate is greater than the threshold and sets a result of the physical condition evaluation to be “OK” when the amount of increase in the heart rate is smaller than or equal to the threshold.

The physical condition evaluation data stores in advance an optimum threshold for each set of values of the combination of the amount of increase in the travel speed and the amount of increase in the altitude (or the amount of decrease in the barometric pressure). Specifically, in the physical condition evaluation data, the greater the amount of increase in the travel speed, the greater the related threshold, and the greater the amount of increase in the altitude (or the amount of decrease in the barometric pressure), the greater the related threshold.

Therefore, for example, even though the route along which the user is traveling is flat and the travel speed remains unchanged, when the heart rate significantly increases, a result of the physical condition evaluation is set to be “NG, ” and even though the route along which the user is traveling is a steep upslope, when the heart rate only slightly increases, a result of the physical condition evaluation is set to be “OK.”

In the physical condition evaluation described above, the order in which the steps described above are carried out can be changed as appropriate. For example, the points of time at which steps S21, S22, and S23 are carried out can be swapped with each other.

### 1-5-1. Heart Rate Threshold

The physical condition evaluation described above is based on the following fact.

A major factor that causes a mountain climber to feel ill may, for example, be altitude sickness. Altitude sickness is a serious disease that endangers the mountain climber's life.

It is known that whether or not a mountain climber has caught altitude sickness can be inferred from the blood oxygen saturation (SpO₂) of the mountain climber. Specifically, the lower the blood oxygen saturation (SpO₂) of the mountain climber, the higher the doubt whether the mountain climber has caught altitude sickness.

Further, a person generally has a function of attempting, when the blood oxygen saturation decreases, to cause the blood oxygen saturation to return to a normal value by unconsciously raising the heart rate (tachycardia reflex).

Further, a person generally has a function of attempting, when the blood oxygen saturation returns to a normal value, to avoid too large an increase in the blood oxygen saturation by unconsciously lowering the heart rate (bradycardia reflex).

It is further known that the speed at which the blood oxygen saturation decreases and the speed at which the heart rate increases are strongly correlated to each other (chemoreceptor reflex sensitivity).

In view of the facts described above, the physical condition evaluation in the present embodiment is performed by inference of a temporal change in the blood oxygen saturation from a temporal change in the heart rate and comparison of the degree of the temporal change with a threshold. Therefore, when a result of the physical condition evaluation is “OK,” the doubt whether a person has caught altitude sickness can be considered to be low, whereas when a result of the physical condition evaluation is “NG,” the doubt whether the person has caught altitude sickness can be considered to be high.

The reflex (tachycardia reflex) occurs relatively quickly when the blood oxygen saturation decreases (probably to avoid life crisis), whereas the reflex (bradycardia reflex) occurs relatively slowly when the blood oxygen saturation increases (probably to assure safety). Therefore, in graphical representation of the bradycardia reflex and the tachycardia reflex described above, a curve representing the characteristic of the bradycardia reflex and a curve representing the characteristic of the tachycardia reflex differ from each other in terms of gradient.

In view of the consideration described above, in the physical condition evaluation in the present embodiment, to appropriately find out both the timing at which a doubt of altitude sickness arises and the timing at which the altitude sickness disappears, a heart rate threshold used when the amount of increase in the heart rate is a positive value and a heart rate threshold used when the amount of increase in the heart rate is a negative value may differ from each other. The thus set heart rate threshold allows the physical condition evaluation to tend to correctly show an evaluation result of “NG” when a doubt of altitude sickness arises and a result of “OK” when the doubt of altitude sickness is cleared.

Further, the relationship between the blood oxygen saturation and whether or not a mountain climber has caught altitude sickness can be determined in advance, for example, on the basis of experimental data showing the relationship between the blood oxygen saturation of the climber and the climber's subjective symptom of altitude sickness or known theoretical data. The heart rate threshold is therefore desirably set in an appropriate manner on the basis of the relationship.

In the physical condition evaluation in the present embodiment, to appropriately set the heart rate threshold, the contents of the physical condition evaluation data only need to be appropriately set in advance. It is therefore assumed in the physical condition evaluation data in the present embodiment that the relationship of the combination of the amount of increase in the travel speed and the amount of increase in the altitude (or the amount of decrease in the barometric pressure) with the heart rate threshold is appropriately set in advance on the basis of experimental data or theoretical data.

### 1-6. Route Search

FIG. 5C is a flowchart for describing route-search-related action of the processor 120. The route search is performed in accordance with the route search program 137. The timing at which the route search is performed is, for example, timing at which a route needs to be searched in individual assistance, which will be described later. Each step in the route search will be sequentially described below.

Step S31: The processor 120 refers to the terrestrial magnetism data outputted from the terrestrial magnetism sensor 111 to recognize the orientation of the user at the current point of time (for example, in which direction a predetermined portion of the electronic apparatus 1 faces).

Step S32: The processor 120 recognizes the coordinates of the horizontal position (latitude and longitude) of the user at the current point of time on the basis of the position coordinates contained in the positioning data outputted from the GPS sensor 110. The processor 120 further calculates the route along which the user has traveled up to the current point of time on the basis of a temporal change in the position coordinates contained in the positioning data outputted from the GPS sensor 110.

The route along which the user has traveled up to the current point of time can be calculated on the basis of the history of the coordinates of the horizontal position (latitude and longitude) of the user. The start point of the travel route has horizontal position coordinates at the point of time when an initiation instruction (which will be described later) is inputted.

Step S33: The processor 120 refers to the atmospheric pressure data outputted from the atmospheric pressure sensor 112 to calculate the altitude of (or the barometric pressure at) the point where the user is present at the current point of time.

Step S34: The processor 120 searches routes stored in the route search data (planned route and reserve route) on the basis of the orientation of the user, the horizontal position coordinates of the user, the travel route up to the current point of time, and the altitude of the point where the user is present to detect a mountain descending route that is shorter than the travel route up to the current point of time and is the shortest of the searched routes (shortest route).

However, if no mountain descending route shorter than the travel route up to the current point of time is detected, the processor 120 detects a route that traces back the travel route up to the current point of time (travel-back route) as the shortest route.

In the route search described above, the order in which the steps described above are carried out can be changed as appropriate. For example, the points of time at which steps S31, S32, and S33 are carried out can be swapped with each other.

### 1-7. Flow of Data

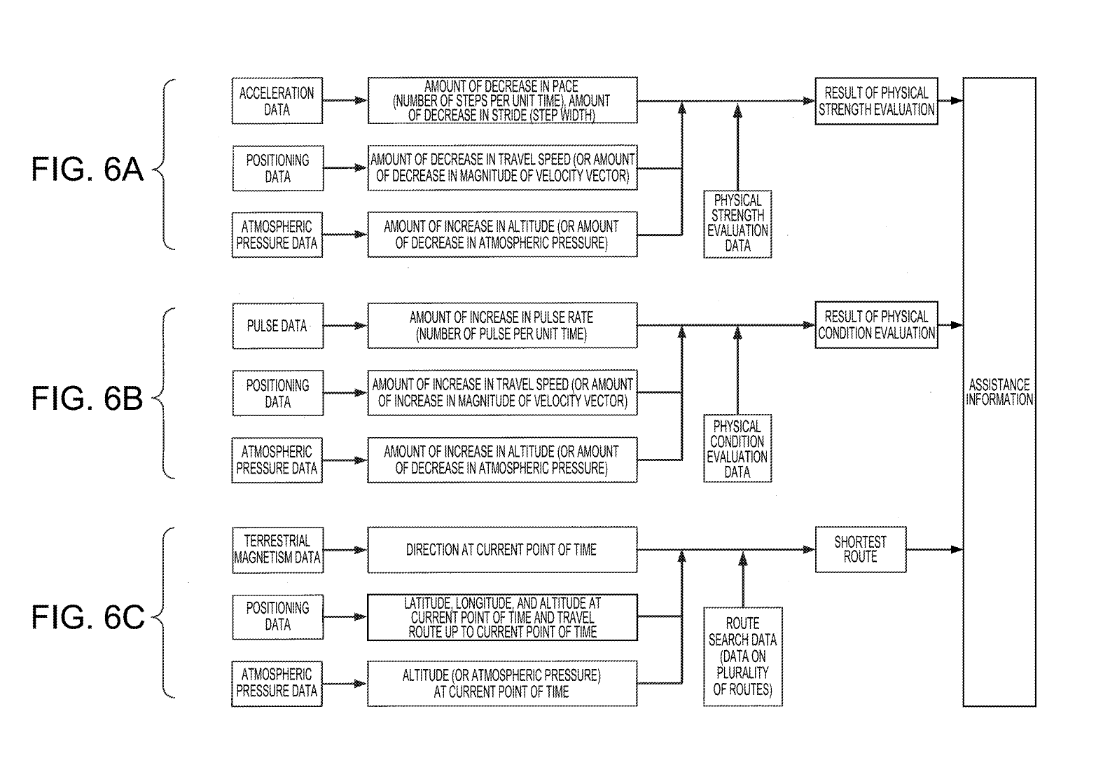

FIG. 6 shows visualized representation of the flow of data in the physical strength evaluation, the physical condition evaluation, and the route search described above.

(A) in FIG. 6 shows the flow of data in the physical strength evaluation. (B) in FIG. 6 shows the flow of data in the physical condition evaluation. (C) in FIG. 6 shows the flow of data in the route search.

A physical strength evaluation result of the physical strength evaluation (FIG. 6(A)), a physical condition evaluation result of the physical condition evaluation (FIG. 6(B)), and a route search result of the route search (shortest route) (FIG. 6(C)) are used, for example, in the individual assistance. In the individual assistance, assistance information is generated on the basis of the physical strength evaluation result, the physical condition evaluation result, and the shortest route, and the assistance information is provided to the user or an external apparatus. The details of the individual assistance will be described below.

### 1-8. Individual Assistance

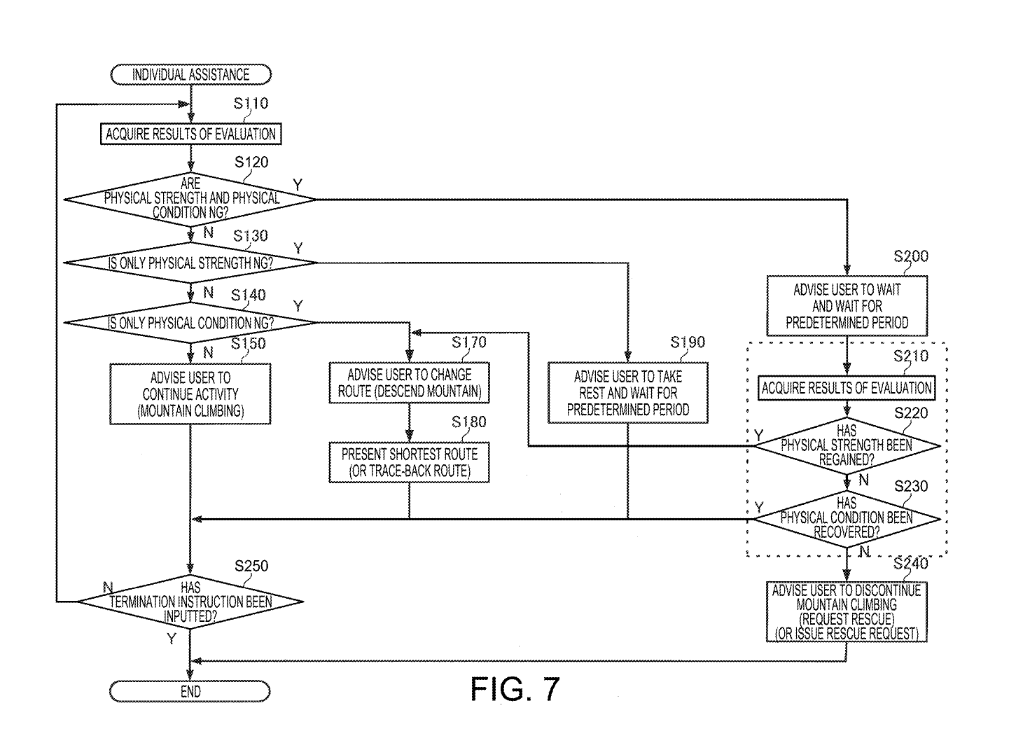

FIG. 7 is a flowchart for describing individual-assistance-related action of the processor 120.

The individual assistance is performed in accordance with the individual assistance program 138. The timing at which the individual assistance is initiated is, for example, timing at which the user inputs an initiation instruction to the electronic apparatus 1 at the start point of the mountain climbing route on the day of the mountain climbing. The timing at which the individual assistance is terminated is, for example, timing at which the user inputs a termination instruction to the electronic apparatus 1 at the goal point of the mountain climbing route on the day of the mountain climbing. The user's input of the initiation instruction and the termination instruction is performed via the input 150. Each step in the individual assistance will be sequentially described below.

Step S110: The processor 120 performs the physical strength evaluation and the physical condition evaluation described above to acquire a result of the evaluation of the user's physical strength and a result of the evaluation of the user's physical condition.

Step S120: The processor 120 evaluates whether or not the result of the physical strength evaluation and the result of the physical condition evaluation are both NG. When a result of the evaluation shows that the results are both NG (Y in step S120), the processor 120 proceeds to step S200, whereas when a result of the evaluation does not show that the results are both NG (N in step S120), the processor 120 proceeds to step S130.

Step S130: The processor 120 evaluates whether or not only the result of the physical strength evaluation, which is one of the result of the physical strength evaluation and the result of the physical condition evaluation, is NG. When a result of the evaluation shows that only the result of the physical strength evaluation is NG (Y in step S130), the processor 120 proceeds to step S190, whereas when a result of the evaluation does not show that only the result of the physical strength evaluation is NG (N in step S130), the processor 120 proceeds to step S140.

Step S140: The processor 120 evaluates whether or not only the result of the physical condition evaluation, which is one of the result of the physical strength evaluation and the result of the physical condition evaluation, is NG. When a result of the evaluation shows that only the result of the physical condition evaluation is NG (Y in step S140), the processor 120 proceeds to step S170, whereas when a result of the evaluation does not show that only the result of the physical condition evaluation is NG (N in step S140), the processor 120 proceeds to step S150.

Step S150: The processor 120 generates assistance information for prompting the user to continue the activity (mountain climbing in the description) and provides the user with the assistance information. For example, the processor 120 generates text image data “Continue activity” and outputs the data to the display 170. The display 170 advises the user to continue the activity by displaying a text image “Continue activity” in accordance with the data inputted from the processor 120. Further, for example, the processor 120 generates audio data “Continue activity” and outputs the data to the audio 180. The audio 180 advises the user to continue the activity by outputting audio “Continue activity” in accordance with the audio data inputted from the processor 120. The processor 120 then proceeds to step S250.

Step S170: The processor 120 generates assistance information for prompting the user to change the route (descend mountain) and provides the user with the assistance information. For example, the processor 120 generates text image data “Descend mountain” and outputs the data to the display 170. The display 170 advises the user to descend the mountain by displaying a text image “Descend mountain” in accordance with the data inputted from the processor 120. Further, for example, the processor 120 generates audio data “Descend mountain” and outputs the data to the audio 180. The audio 180 advises the user to descend the mountain by outputting audio “Descend mountain” in accordance with the audio data inputted from the processor 120.

Step S180: The processor 120 performs the route search described above and presents the user the shortest route (or trace-back route) detected in the route search. For example, the processor 120 generates image data of a curved line or a zigzag line representing the shortest route and outputs the data to the display 170. The display 170 displays an image of the shortest route in accordance with the data inputted from the processor 120. The processor 120 then proceeds to step S250.

Step S190: The processor 120 generates assistance information for prompting the user to temporarily stop the mountain climbing (take a rest) and provides the user with the assistance information. For example, the processor 120 generates text image data “Take rest” and outputs the data to the display 170. The display 170 advises the user to take a rest by displaying a text image “Take rest” in accordance with the data inputted from the processor 120. Further, the processor 120 generates audio data “Take rest” and outputs the data to the audio 180. The audio 180 advises the user to take a rest by outputting audio “Take rest” in accordance with the audio data inputted from the processor 120. The processor 120 then waits for a predetermined period (10 minutes, for example) and proceeds to step S250.

Step S200: The processor 120 generates assistance information for prompting the user to wait at the current point and provides the user with the assistance information. For example, the processor 120 generates text image data “Wait” and outputs the data to the display 170. The display 170 advises the user to wait by displaying a text image “Wait” in accordance with the data inputted from the processor 120. Further, the processor 120 generates audio data “Wait” and outputs the data to the audio 180. The audio 180 advises the user to wait by outputting audio “Wait” in accordance with the audio data inputted from the processor 120. The processor 120 then waits for a predetermined period (10 minutes, for example) and proceeds to step S210.

Step S210: The processor 120 performs the physical strength evaluation and the physical condition evaluation again to acquire a result of the physical strength evaluation and a result of the physical condition evaluation.

Step S220: The processor 120 evaluates whether or not the result of the evaluation of the user's physical strength has returned to OK (whether or not physical strength has been regained). When a result of the evaluation shows that the physical strength has been regained (Y in step S220), the processor 120 proceeds to step S170, whereas when a result of the evaluation does not show that the physical strength has been regained (N in step S220), the processor 120 proceeds to step S230.

Step S230: The processor 120 evaluates whether or not the result of the evaluation of the user's physical condition has returned to OK (whether or not physical condition has been recovered). When a result of the evaluation shows that the physical condition has been recovered (Y in step S230), the processor 120 proceeds to step S250, whereas when a result of the evaluation does not show that the physical condition has been recovered (N in step S230), the processor 120 proceeds to step S240.

Step S240: The processor 120 generates assistance information for prompting the user to discontinue the mountain climbing (request rescue) and provides the user with the assistance information. For example, the processor 120 generates text image data “Request rescue” and outputs the data to the display 170. The display 170 advises the user to discontinue the mountain climbing by displaying a text image “Request rescue” in accordance with the data inputted from the processor 120. Further, the processor 120 generates audio data “Request rescue” and outputs the data to the audio 180. The audio 180 advises the user to discontinue the mountain climbing by outputting audio “Request rescue” in accordance with the audio data inputted from the processor 120 and terminates the procedure of the flowchart.

The processor 120 in step S240 may issue rescue request directly to an external apparatus in place of or in addition to advising the user to discontinue the mountain climbing in consideration of a possibility of a situation in which the user is unable to request rescue by himself/herself (for example, a case where the user is unconscious or the user's hands or fingers are paralyzed). The “external apparatus” used herein is an apparatus that can communicate with the electronic apparatus 1 via the communication device 190 thereof and may include at least one of the electronic apparatus 1 of another user, a communication apparatus installed in a police station or a fire department, and a communication apparatus carried by a rescue team.

The processor 120 in step S240 may issue a distress signal by itself in place of or in addition to the rescue request. The distress signal is formed, for example, of a sound wave (audible sound, ultrasonic wave), an electromagnetic wave (visible light, radio wave), vibration, or a combination of at least two of the forms described above.

The distress signal formed of visible light can be issued, for example, via the display 170. The distress signal formed of an electromagnetic wave can be issued, for example, via the communication device 190. The distress signal formed of a sound wave can be issued, for example, via the audio 180.

Step S250: The processor 120 evaluates whether or not the user has inputted the termination instruction via the input 150. When a result of the evaluation shows that the user has not inputted the termination instruction (N in step S250), the processor 120 proceeds to step S110, whereas when a result of the evaluation shows that the user has inputted the termination instruction (Y in step S250), the processor 120 terminates the procedure of the flowchart.

In the individual assistance described above, part of the steps can be swapped with each other in terms of the order in which they are carried out, or part of the steps can be omitted. For example, steps S210, S220, and S230, which are surrounded by the dotted-line frame in FIG. 7, can be omitted.

### 2-1. Increasing the Number of Electronic Apparatus

It is assumed in the above description that the electronic apparatus 1 assists a single mountain climber. Instead, two or more electronic apparatus 1 can form a system (system formed of an increased number of electronic apparatus 1) and the system can assist a mountain climbing party. The mountain climbing party refers to a party (team, group) formed of a plurality of participants who take part together in mountain climbing. In the following description, the same configurations as those in the above description have the same reference characters and will not be described in detail.

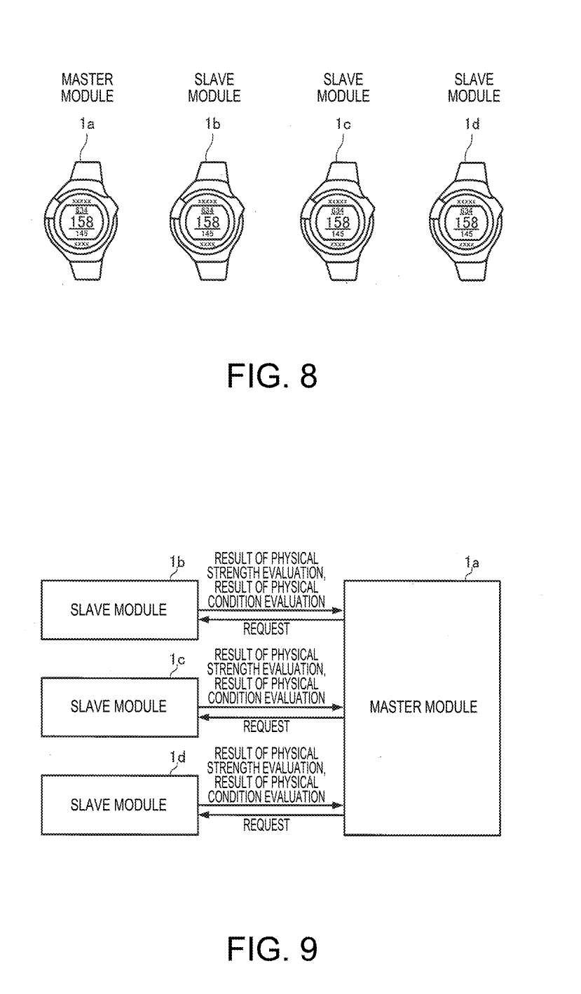

FIG. 8 describes a system that assists a mountain climbing party. It is assumed in the following description that the number of participants in the mountain climbing party is “4”, the electronic apparatus 1 carried by a leader of the mountain climbing party is used as a master module 1a, and the electronic apparatus 1 carried by the three participants (hereinafter referred to as “climbing companions”) in the mountain climbing party other than the leader are used as slave modules 1b, 1c, and 1d.

Before the mountain climbing, the leader or any of the climbing companions in the mountain climbing party operates the input 150 of the electronic apparatus 1 carried by the leader to input a predetermined signal to the electronic apparatus 1 so as to cause the electronic apparatus 1 to recognize that the electronic apparatus 1 is used as the master module 1a.

The master module 1a is a module that collects necessary information from the slave modules 1b, 1c, and 1d to assist the entire mountain climbing party. The master module 1a provides a function of performing the physical strength evaluation described above, a function of performing the physical condition evaluation described above, a function of performing the route search described above, and a function of performing party assistance, which will be described below.

Further, before the mountain climbing, the leader or any of the climbing companions in the mountain climbing party operates the input 150 of the master module 1a to input a predetermined signal to the master module 1a so as to cause the electronic apparatus 1 carried by one of the three climbing companions to communicate with the master module 1a at least once so that both the electronic apparatus 1 and the master module 1a recognize that the electronic apparatus 1 is used as the slave module 1b.

Still further, before the mountain climbing, the leader or any of the climbing companions in the mountain climbing party operates the input 150 of the master module 1a to input a predetermined signal to the master module 1a so as to cause the electronic apparatus 1 carried by another of the three climbing companions to communicate with the master module 1a at least once so that both the electronic apparatus 1 and the master module 1a recognize that the electronic apparatus 1 is used as the slave module 1c.

Still further, before the mountain climbing, the leader or any of the climbing companions in the mountain climbing party operates the input 150 of the master module 1a to input a predetermined signal to the master module 1a so as to cause the electronic apparatus 1 carried by the remaining one of the three climbing companions to communicate with the master module 1a at least once so that both the electronic apparatus 1 and the master module 1a recognize that the electronic apparatus 1 is used as the slave module 1d.

Each of the slave modules 1b, 1c, and 1d is a module that transmits information to the master module 1a in response to a request from the master module 1a. Each of the slave modules 1b, 1c, and 1d provides the function of performing the physical strength evaluation described above and the function of performing the physical condition evaluation described above.

The communication between the master module 1a and each of the slave modules 1b, 1c, and 1d is performed via the processor 120 and the communication device 190 incorporated in the master module 1a and the processor 120 and the communication device 190 incorporated in each of the slave modules 1b, 1c, and 1d (the same holds true for the following description).

In the party assistance during the mountain climbing, the master module 1a requests as required each of the slave modules 1b, 1c, and 1d to transmit a result of the physical strength evaluation and a result of the physical condition evaluation, as shown in FIG. 9. Each of the slave modules 1b, 1c, and 1d performs the physical strength evaluation and the physical condition evaluation in response to the request. Each of the slave modules 1b, 1c, and 1d then transmits a result of the physical strength evaluation and a result of the physical condition evaluation to the master module 1a with identification information on the slave module (slave module identification information) attached to the results.

The master module 1a can therefore acquire (collect) the result of the physical strength evaluation and the result of the physical condition evaluation from each of the slave modules 1b, 1c, and 1d. The evaluation results collected by the master module 1a are the following six types of evaluation result.

(1) A result of the physical strength evaluation associated with the climbing companion who carries the slave module 1b

(2) A result of the physical condition evaluation associated with the climbing companion who carries the slave module 1b

(3) A result of the physical strength evaluation associated with the climbing companion who carries the slave module 1c

(4) A result of the physical condition evaluation associated with the climbing companion who carries the slave module 1c

(5) A result of the physical strength evaluation associated with the climbing companion who carries the slave module 1d

(6) A result of the physical condition evaluation associated with the climbing companion who carries the slave module 1d

### 2-2. Party Assistance

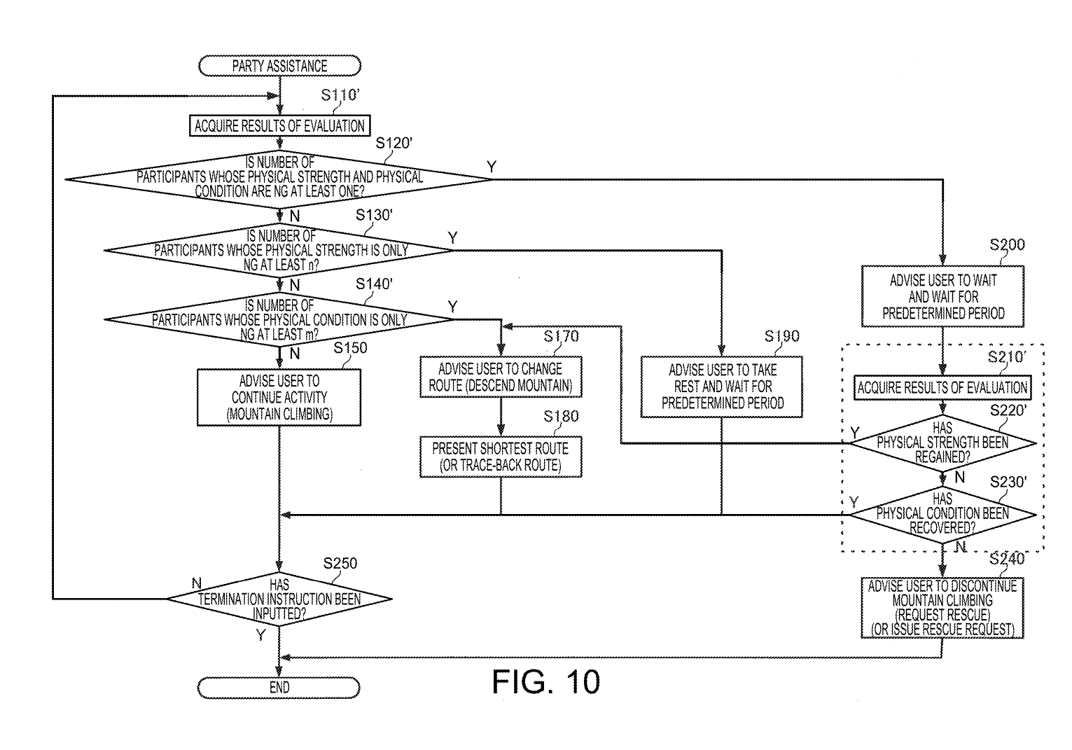

FIG. 10 is a flowchart for describing party-assistance-related action of the master module 1a (action of processor 120 of master module 1a).

The party assistance (example of physical activity assistance method) is performed in accordance with the party assistance program 139 (example of physical activity assistance program). The timing at which the party assistance is initiated is, for example, timing at which the leader inputs the initiation instruction to the master module 1a at the start point of the mountain climbing route on the day of the mountain climbing. The timing at which the party assistance is terminated is, for example, timing at which the leader inputs the termination instruction to the master module 1a at the goal point of the mountain climbing route on the day of the mountain climbing. (The leader's input of the initiation instruction and the termination instruction is performed via the input 150 of the master module 1a.) Each step in the party assistance will be sequentially described below.

Step S110′: The processor 120 of the master module 1a performs the physical strength evaluation and the physical condition evaluation described above to acquire a result of the physical strength evaluation and a result of the physical condition evaluation associated with the leader. The processor 120 of the master module 1a further requests each of the slave modules 1b, 1c, and 1d to transmit a result of the physical strength evaluation and a result of the physical condition evaluation and collects the result of the physical strength evaluation and the result of the physical condition evaluation associated with each of the three climbing companions.

Step S120′: The processor 120 of the master module 1a evaluates whether or not the number of participants whose results of the physical strength evaluation and the physical condition evaluation are both NG is at least one. When a result of the evaluation shows that the number is at least one (Y in Step S120′), the processor 120 proceeds to step S200, whereas when a result of the evaluation does not show that the number is at least one (N in Step S120′), the processor 120 proceeds to step S130′.

Step S130′: The processor 120 of the master module 1a evaluates whether or not the number of participants whose result of the physical strength evaluation, which is one of the result of the physical strength evaluation and the result of the physical condition evaluation, is only NG is at least n. When a result of the evaluation shows that the number is at least n (Y in Step S130′), the processor 120 proceeds to step S190, whereas when a result of the evaluation does not show that the number is at least n (N in Step S130′), the processor 120 proceeds to step S140′.

The number n is a threshold appropriately set by the processor 120 of the master module 1a in accordance with the number of participants (“4” in the description) (for example, n is set at about half of the number of participants).

Step S140′: The processor 120 of the master module 1a evaluates whether or not the number of participants whose result of the physical condition evaluation, which is one of the result of the physical strength evaluation and the result of the physical condition evaluation, is only NG is at least m. When a result of the evaluation shows that the number is at least m (Y in Step S140′), the processor 120 proceeds to step S170, whereas when a result of the evaluation does not show that the number is at least m (N in Step S140′), the processor 120 proceeds to step S150.

The number m is a threshold appropriately set by the processor 120 of the master module 1a in accordance with the number of participants (“4” in the description) (for example, m is set at about half of the number of participants).

Step S150: The processor 120 of the master module 1a generates assistance information for prompting the party to continue the activity (mountain climbing in the description) and provides the leader with the assistance information. For example, the processor 120 of the master module 1a generates text image data “Continue activity” and outputs the data to the display 170. The display 170 advises the leader to continue the activity by displaying a text image “Continue activity” in accordance with the data inputted from the processor 120. Further, for example, the processor 120 of the master module 1a generates audio data “Continue activity” and outputs the data to the audio 180. The audio 180 advises the leader to continue the activity by outputting audio “Continue activity” in accordance with the audio data inputted from the processor 120. The processor 120 of the master module 1a then proceeds to step S250.

Step S170: The processor 120 of the master module 1a generates assistance information for prompting the party to change the route (descend mountain) and provides the user (leader) with the assistance information. For example, the processor 120 of the master module 1a generates text image data “Descend mountain” and outputs the data to the display 170. The display 170 advises the leader to descend the mountain by displaying a text image “Descend mountain” in accordance with the data inputted from the processor 120. Further, for example, the processor 120 of the master module 1a generates audio data “Descend mountain” and outputs the data to the audio 180. The audio 180 advises the leader to descend the mountain by outputting audio “Descend mountain” in accordance with the audio data inputted from the processor 120.

Step S180: The processor 120 of the master module 1a performs the route search described above and presents the leader the shortest route (or trace-back route) detected in the route search. For example, the processor 120 of the master module 1a generates image data of a curved line or a zigzag line representing the shortest route and outputs the image data to the display 170. The display 170 displays an image of the shortest route in accordance with the data inputted from the processor 120. The processor 120 of the master module 1a then proceeds to step S250.

Step S190: The processor 120 of the master module 1a generates assistance information for prompting the party to temporarily stop the mountain climbing (take a rest) and provides the leader with the assistance information. For example, the processor 120 of the master module 1a generates text image data “Take rest” and outputs the data to the display 170. The display 170 advises the leader to take a rest by displaying a text image “Take rest” in accordance with the data inputted from the processor 120. Further, the processor 120 of the master module 1a generates audio data “Take rest” and outputs the data to the audio 180. The audio 180 advises the leader to take a rest by outputting audio “Take rest” in accordance with the audio data inputted from the processor 120. The processor 120 of the master module 1a then waits for a predetermined period (10 minutes, for example) and proceeds to step S250.

Step S200: The processor 120 of the master module 1a generates assistance information for prompting the party to wait at the current point and provides the leader with the assistance information. For example, the processor 120 of the master module 1a generates text image data “Wait” and outputs the data to the display 170. The display 170 advises the leader to wait by displaying a text image “Wait” in accordance with the data inputted from the processor 120. Further, the processor 120 of the master module 1a generates audio data “Wait” and outputs the data to the audio 180. The audio 180 advises the leader to wait by outputting audio “Wait” in accordance with the audio data inputted from the processor 120. The processor 120 of the master module 1a then waits for a predetermined period (10 minutes, for example) and proceeds to step S210′.

Step S210′: The processor 120 of the master module 1a performs the physical strength evaluation and the physical condition evaluation again to acquire a result of the physical strength evaluation and a result of the physical condition evaluation associated with the leader. The processor 120 of the master module 1a further requests each of the slave modules 1b, 1c, and 1d again to transmit a result of the physical strength evaluation and a result of the physical condition evaluation and collects the result of the physical strength evaluation and the result of the physical condition evaluation associated with each of the three climbing companions.

Step S220′: The processor 120 of the master module 1a evaluates whether or not the result of the physical strength evaluation associated with each of the users whose results of the physical strength evaluation and the physical condition evaluation were both NG has returned to OK (whether or not physical strength has been regained). When a result of the evaluation shows that the physical strength has been regained (Y in step S220′), the processor 120 proceeds to step S170, whereas when a result of the evaluation does not show that the physical strength has been regained (N in step S220′), the processor 120 proceeds to step S230′.

Step S230′: The processor 120 of the master module 1a evaluates whether or not the result of the physical condition evaluation associated with each of the users whose results of the physical strength evaluation and the physical condition evaluation were both NG has returned to OK (whether or not physical condition has been recovered). When a result of the evaluation shows that the physical condition has been recovered (Y in step S230′), the processor 120 proceeds to step S250, whereas when a result of the evaluation does not show that the physical condition has been recovered (N in step S230′), the processor 120 proceeds to step S240.

Step S240: The processor 120 of the master module 1a generates assistance information for prompting the party to discontinue the mountain climbing (request rescue) and provides the leader with the assistance information. For example, the processor 120 of the master module 1a generates text image data “Request rescue” and outputs the data to the display 170. The display 170 advises the leader to discontinue the mountain climbing by displaying a text image “Request rescue” in accordance with the data inputted from the processor 120. Further, the processor 120 of the master module 1a generates audio data “Request rescue” and outputs the data to the audio 180. The audio 180 advises the leader to discontinue the mountain climbing by outputting audio “Request rescue” in accordance with the audio data inputted from the processor 120 and terminates the procedure of the flowchart.

The processor 120 of the master module 1a in step S240 may issue rescue request directly to an external apparatus in place of or in addition to advising the leader to discontinue the mountain climbing in consideration of a possibility of a situation in which the leader is unable to request rescue by himself/herself (for example, a case where the leader is unconscious or the leader's hands or fingers are paralyzed). The “external apparatus” used herein is an apparatus that can communicate with the master module 1a via the communication device 190 thereof and may include at least one of the electronic apparatus 1 of another user, a communication apparatus installed in a police station or a fire department, a communication apparatus carried by a rescue team, and slave modules 1b, 1c, and 1d.

The processor 120 of the master module 1a in step S240 may further issue a distress signal by itself in place of or in addition to the rescue request. The distress signal is formed, for example, of a sound wave (audible sound, ultrasonic wave), an electromagnetic wave (visible light, radio wave), vibration, or a combination of at least two of the forms described above.

The distress signal formed of visible light can be issued, for example, via the display 170. The distress signal formed of an electromagnetic wave can be issued, for example, via the communication device 190. The distress signal formed of a sound wave can be issued, for example, via the audio 180.

Step S250: The processor 120 of the master module 1a evaluates whether or not the user has inputted the termination instruction via the input 150. When a result of the evaluation shows that the user has not inputted the termination instruction (N in step S250), the processor 120 proceeds to step S110′, whereas when a result of the evaluation shows that the user has inputted the termination instruction (Y in step S250), the processor 120 terminates the procedure of the flowchart.

In the party assistance described above, part of the steps can be swapped with each other in terms of the order in which they are carried out, or part of the steps can be omitted. For example, steps S210′, S220′, and S230′, which are surrounded by the dotted-line frame in FIG. 10, can be omitted.

### 3. Supplementary Description of Embodiment

In the physical condition evaluation described above, the user's blood oxygen saturation is indirectly detected from the user's heart rate, but the user's blood oxygen saturation may instead be directly detected.

In the physical condition evaluation described above, the user's heart rate is indirectly detected from the number of pulses in the user's arm and may instead be detected from the number of pulses in a site other than the user's arm. For example, a heart rate sensor (which is attached to the user's chest with a belt) may be used in place of the pulse sensor.

In the physical condition evaluation described above, the physical condition evaluation is performed on the basis of a temporal change in the user's heart rate and may instead be performed on the basis of a temporal change in biological information other than the heart rate. For example, the physical condition evaluation may instead be performed on the basis of the user's respiration rate, blood sugar level, sweat rate, body temperature, blood salinity concentration, cardiograph, electromyogram, or information, for example, on the brain wave.

In the route search described above, the shortest route is detected as a route along which the user descends the mountain. Instead, a route over which the user can travel in the shortest expected period or a route that imposes the lightest burden on the user (for example, a route with a small number of ups and downs and a route including no steep slope) may be detected.

In the route search described above, the route search data stored in advance in the memory 130 of the electronic apparatus 1 is used as a database. Instead, in a case where the electronic apparatus 1 is connectable to a network, such as the Internet, a database stored in a network server or any other component may be used. Further, a method for searching for a route may be any known method.

In the physical condition evaluation described above, altitude sickness is presented by way of example, and other diseases that are likely to force reconsideration of the mountain climbing route, such as fever, low body temperature, a low blood sugar level, and heat stroke, may be taken into consideration.

The thresholds in the physical strength evaluation or the physical condition evaluation described above may be adjusted as appropriate in accordance with the user's attributes (such as age, sex, whether or not the user is an athlete) inputted to the electronic apparatus 1 in advance or may be adjusted by the user himself/herself.

For example, in the physical strength evaluation or the physical condition evaluation described above, the thresholds in a case where the user's age belongs to a young generation may be set to be higher than the thresholds in a case where the user's age belongs to an elderly generation (that is, the thresholds may be so set that a result of the evaluation is more unlikely to be evaluated to be NG).

For example, in the physical strength evaluation or the physical condition evaluation described above, the thresholds in a case where the user is an athlete may be set to be higher than the thresholds in a case where the user is not an athlete (that is, the thresholds may be so set that a result of the evaluation is more unlikely to be evaluated to be NG).

In step S190 in the party assistance described above, the assistance information for prompting the party to take a rest in the mountain climbing. Instead, assistance information for prompting any of the participants to help another out instead of prompting the party to take a rest.

The term “help” used herein is what is called helping out each other, for example, a participant whose result of the physical strength evaluation is OK takes over part of the burden on a participant whose result of the physical strength evaluation is NG.

Instead, in step S190 in the party assistance described above, the assistance information for prompting participants to help out may be generated when the number of participants whose results of the physical strength evaluation are NG exceeds a first threshold, and the assistance information for prompting the party to “take a rest” may be generated when the number of participants whose results of the physical strength evaluation are NG exceeds a second threshold greater than the first threshold.

The processor 120 in the embodiment described above primarily uses the GPS sensor 110 as a position sensor for calculating the coordinates of the position of the electronic apparatus 1 and may instead use at least one of the GPS sensor 110, the terrestrial magnetism sensor 111, the atmospheric pressure sensor 112, the acceleration sensor 113, and the angular velocity sensor 114.

### 4. Outline of Embodiment

(1) The electronic apparatus 1 according to the present embodiment includes the processor 120, which generates, on the basis of the evaluation of the physical strength (steps S11 to S15) of the user who is performing physical activity and the evaluation of the physical condition (steps S21 to S25) of the user, information (assistance information) on whether or not the user's physical activity needs to be reconsidered.

That is, the processor 120 generates the information (assistance information) on the basis of both the user's physical strength and physical condition to reflect both the user's physical strength and physical condition in the information (assistance information). The user can therefore use the information (assistance information) as a supplement for making appropriate judgement during the physical activity.

(2) In the electronic apparatus 1 according to the present embodiment, the processor 120 performs the physical strength evaluation (steps S11 to S15) on the basis of a temporal change in the user's physical activity.

That is, the processor 120 uses a temporal change in the user's physical activity performance (instead of the user's physical activity performance itself). A decrease in the user's physical strength or any other parameter over time can therefore be reflected in a result of the physical strength evaluation.

(3) In the electronic apparatus 1 according to the present embodiment, the processor 120 performs the physical strength evaluation (steps S11 to S15) on the basis of a temporal change in at least one of the altitude of the point where the user is present, the number of user's steps (pace), the user's speed (travel speed), the user's step width (stride), and the barometric pressure at the point where the user is present.

That is, the processor 120 uses a temporal change in at least one of the altitude, the number of steps (pace), the speed (travel speed), and the step width (stride) as an index of the physical strength evaluation (steps S11 to S15). These items tend to change, for example, as the physical strength of the user who is performing the physical activity changes. The user's physical strength can therefore be reflected in a result of the physical strength evaluation.

(4) In the electronic apparatus 1 according to the present embodiment, the processor 120 performs the physical strength evaluation (steps S11 to S13) on the basis of an output from at least one of a position sensor, an acceleration sensor, and an atmospheric pressure sensor.

That is, the processor 120 can use the output from at least one of the position sensor, the acceleration sensor, and the atmospheric pressure sensor in the physical strength evaluation (steps S11 to S15).

(5) In the electronic apparatus 1 according to the present embodiment, the processor 120 performs the physical condition evaluation (steps S21 to S25) on the basis of a temporal change in the user's biological information.

That is, the processor 120 uses a temporal change in the user's biological information (instead of user's physical condition itself) in the physical condition evaluation (steps S21 to S25). For example, decline in the user's physical condition over time can therefore be reflected in a result of the physical condition evaluation.

(6) In the electronic apparatus 1 according to the present embodiment, the processor 120 performs the physical condition evaluation (steps S21 to S25) on the basis of a temporal change in the user's heart rate.

That is, the processor 120 can reflect a temporal change in the heart rate (decline in user's physical condition (for example, whether or not the user has caught altitude sickness)) in a result of the physical condition evaluation.

(7) In the electronic apparatus 1 according to the present embodiment, the processor 120 generates information representing that the physical activity does not need to be reconsidered (assistance information for prompting user to continue physical activity) when a result of the physical strength evaluation (steps S11 to S15) and a result of the physical condition evaluation (steps S21 to S25) are both OK.

That is, the processor 120 generates information representing that the physical activity does not need to be reconsidered (assistance information for prompting user to continue physical activity) when a result of the evaluation of the user's physical strength and a result of the evaluation of the user's physical condition are OK. The information (assistance information for prompting user to continue physical activity) can prompt the user to continue the physical activity.

(8) In the electronic apparatus 1 according to the present embodiment, the processor 120 generates information representing that the physical activity needs to be changed (assistance information for prompting user to change route) (step S170) when, out of a result of the physical strength evaluation (steps S11 to S15) and a result of the physical condition evaluation (steps S21 to S25), only the result of the physical condition evaluation is NG.

That is, the processor 120 generates information representing that the physical activity needs to be changed (assistance information for prompting user to change route) when a result of the evaluation of the user's physical strength is not NG but a result of the evaluation of the user's physical condition is NG. The information (assistance information for prompting user to change route) can prompt the user to change the physical activity.

(9) In the electronic apparatus 1 according to the present embodiment, the processor 120 generates information representing that the physical activity needs to be temporarily stopped (assistance information for prompting user to temporarily stop physical activity) (step S190) when, out of a result of the physical strength evaluation (steps S11 to S15) and a result of the physical condition evaluation (steps S21 to S25), only the result of the physical strength evaluation is NG.

That is, the processor 120 generates information representing that the physical activity needs to be temporarily stopped (assistance information for prompting user to temporarily stop physical activity) when a result of the evaluation of the user's physical strength is NG but a result of the evaluation of the user's physical condition is not NG. The information (assistance information for prompting user to temporarily stop physical activity) can prompt the user to temporarily stop the physical activity.

(10) In the electronic apparatus 1 according to the present embodiment, the processor 120 generates information representing that the physical activity needs to be discontinued (assistance information for prompting user to discontinue physical activity) (step S240) when a result of the physical strength evaluation (steps S11 to S15) and a result of the physical condition evaluation (steps S21 to S25) are both NG.

That is, the processor 120 generates information representing that the physical activity needs to be discontinued (assistance information for prompting user to discontinue physical activity) when a result of the evaluation of the user's physical strength is NG and a result of the evaluation of the user's physical condition is also NG. The information (assistance information for prompting user to discontinue physical activity) can prompt the user to discontinue the physical activity.

(11) In the electronic apparatus 1 according to the present embodiment, the processor 120 generates information on whether or not a plurality of users' physical activity needs to be reconsidered on the basis of the evaluation of the plurality of users' physical strength (steps S11 to S15) and the evaluation of the plurality of users' physical condition (steps S21 to S25) (FIG. 10).

That is, the processor 120 generates information (assistance information) on whether or not the physical activity needs to be reconsidered on the basis of both the plurality of users' physical strength and physical condition to reflect both the plurality of users' physical strength and physical condition in the information (assistance information). The plurality of user can therefore use the information (assistance information) as a supplement for making appropriate judgement during the physical activity.

(12) In the electronic apparatus 1 according to the present embodiment, the processor 120 generates information representing that the physical activity does not need to be reconsidered (assistance information for prompting plurality of users to continue physical activity) (step S150) when results of the evaluation of all the plurality of users' physical strength (steps S11 to S15) and results of the evaluation of all the plurality of users' physical condition (steps S21 to S25) are both OK.

That is, the processor 120 generates information representing that the physical activity does not need to be reconsidered (assistance information for prompting plurality of users to continue physical activity) when results of the evaluation of all the plurality of users' physical strength and results of the evaluation of all the plurality of users' physical condition are OK. The information can prompt the plurality of users to continue the physical activity.

(13) In the electronic apparatus 1 according to the present embodiment, the processor 120 generates information representing that the physical activity needs to be changed (assistance information for prompting plurality of users to change route) (step S170) when the number of users whose results of the physical condition evaluation, out of results of the physical strength evaluation (steps S11 to S15) and results of the physical condition evaluation (steps S21 to S25), are only NG is greater than or equal to a predetermined value.

That is, the processor 120 generates information representing that the physical activity needs to be changed (assistance information for prompting plurality of users to change route) when the number of users whose results of the physical strength evaluation are not NG but results of the physical condition evaluation are NG is greater than or equal to a predetermined value. The information (assistance information for prompting plurality of users to change route) can prompt the users to change the physical activity.

(14) In the electronic apparatus 1 according to the present embodiment, the processor 120 generates information representing that the physical activity needs to be temporarily stopped (assistance information for prompting plurality of users to temporarily stop physical activity) (step S190) when the number of users whose results of the physical strength evaluation, out of results of the physical strength evaluation (steps S11 to S15) and results of the physical condition evaluation (steps S21 to S25), are only NG is greater than or equal to a predetermined value.

That is, the processor 120 generates information representing that the physical activity needs to be temporarily stopped (assistance information for prompting plurality of users to temporarily stop physical activity) when the number of users whose results of the physical strength evaluation are NG but results of the physical condition evaluation are not NG is greater than or equal to a predetermined value. The information (assistance information for prompting plurality of users to temporarily stop physical activity) can prompt the users to temporarily stop the physical activity.

(15) In the electronic apparatus 1 according to the present embodiment, the processor 120 generates information representing that the physical activity needs to be discontinued (assistance information for prompting plurality of users to discontinue physical activity) (step S240) when the number of users whose results of the physical strength evaluation and results of the physical condition evaluation are both NG is greater than or equal to a predetermined value (in the example described above, when the number of users that meet the condition described above exceeds “1”).

That is, the processor 120 generates information representing that the physical activity needs to be discontinued (assistance information for prompting plurality of users to discontinue physical activity) when the number of users whose results of the physical strength evaluation and the physical condition evaluation are NG is greater than or equal to a predetermined value (in the example described above, when the number of users that meet the condition described above exceeds “1”). The information (assistance information for prompting plurality of users to discontinue physical activity) can prompt the users to discontinue the physical activity.

(16) In the electronic apparatus 1 according to the present embodiment, the processor 120 provides the user with information (assistance information) (steps S150, S170, S180, S190, S200, and S240).

That is, the processor 120, for example, can provide information (for example, information on a decrease in the physical strength or decline in the physical condition) on a certain user to the user himself/herself or another user. The processor 120 therefore also allows the user himself/herself or the other user to make objective judgement.

(17) The electronic apparatus 1 according to the present embodiment is attachable to a predetermined site of the user.

The user can therefore use the electronic apparatus 1 without holding the electronic apparatus by a hand (in a hands-free state).

(18) In the electronic apparatus 1 according to the present embodiment, the predetermined site is an armor a wrist.

The user can therefore use the electronic apparatus 1 as if it were, for example, a wristwatch.

5. Other variations

The invention is not limited to the embodiment described above, and a variety of variations are conceivable within the scope of the substance of the invention. In the following description, the same configurations as those in the embodiment described above have the same reference characters and will not be described in detail.

For example, at least part of the functions of the electronic apparatus 1 (for example, function of at least one of physical strength evaluation, physical condition evaluation, route search, individual assistance, and party assistance) may be embodied when a program (software) is executed by a computer (processor 120) incorporated in the electronic apparatus 1 or may be embodied when a processing circuit (hardware) incorporated in the electronic apparatus 1 operates.

Among the functions of the electronic apparatus 1, the functions other than the sensing functions may be provided in an external apparatus that can communicate with the electronic apparatus 1 (for example, smartphone, tablet PC and other mobile terminals, and network server).

The electronic apparatus 1 may be provided with known functions of a smartphone, for example, a camera function, a call function, and a communication function.

Examples of application of the electronic apparatus 1 may include skiing (such as cross-country skiing), running, bicycling, walking, swimming, golf, motorcycling, motorsport, sailing, trail running, paragliding, kite flying, and dogsledding as well as mountain climbing.

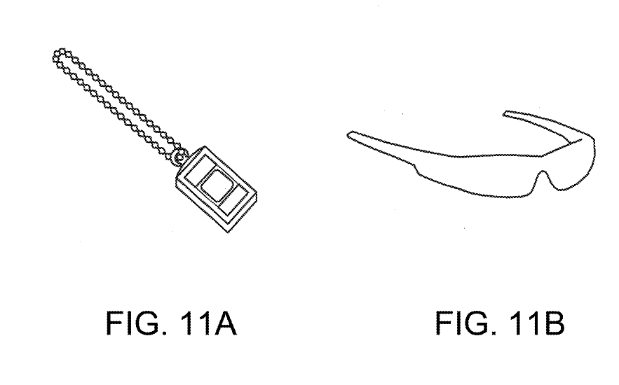

The electronic apparatus 1 can be configured as a wrist-worn-type electronic apparatus, an earphone-type electronic apparatus, a finger-ring-type electronic apparatus, a pendant-type electronic apparatus (see FIG. 11A), an electronic apparatus attached to a sport gear for use, a smartphone, a head mounted display (HMD) (see FIG. 11B), and a variety of other types of mobile information apparatus. When a pulse sensor is incorporated in the electronic apparatus 1, the electronic apparatus 1 is desirably configured to be in contact with the user's body.

The electronic apparatus 1 or the information terminal may notify the user of information in the form of image display, audio, vibration, or any other type of action or a combination of at least two of the image display, audio, and vibration.

In the embodiment described above, a GPS (global positioning system) is used, and a global navigation satellite system (GLASS) may instead be used. For example, one of or two or more of EGNOS (European Geostationary-Satellite Navigation Overlay Service), QZSS (Quasi Zenith Satellite System), GLONASS (GLObal NAvigation Satellite System), GALILEO, BeiDou (BeiDou Navigation Satellite System), and other satellite positioning systems may be used. Further, WAAS (Wide Area Augmentation System), EGNOS (European Geostationary-Satellite Navigation Overlay Service), or any other satellite-based augmentation system (SBAS) may be used as at least one of the satellite positioning systems.

The embodiment and the variations described above are presented by way of example, and the invention is not limited thereto. For example, the embodiment and any of the variations can be combined with each other as appropriate.

The invention encompasses substantially the same configuration as the configuration described in the embodiment (for example, a configuration having the same function, using the same method, and providing the same result or a configuration having the same purpose and providing the same effect). Further, the invention encompasses a configuration in which an inessential portion of the configuration described in the embodiment is replaced. Moreover, the invention encompasses a configuration that provides the same advantageous effects as those provided by the configuration described in the embodiment or a configuration that can achieve the same purpose as that achieved by the configuration described in the embodiment. Further, the invention encompasses a configuration in which a known technology is added to the configuration described in the embodiment.

What is claimed is:

1. An electronic apparatus comprising a processor that generates, on the basis of evaluation of physical strength of a user who is performing physical activity and evaluation of the user's physical condition, information on whether or not the user's physical activity needs to be reconsidered.

2. The electronic apparatus according to claim 1,
wherein the processor performs the physical strength evaluation on the basis of a temporal change in the user's physical activity.

3. The electronic apparatus according to claim 2,
wherein the processor performs the physical strength evaluation on the basis of a temporal change in at least one of altitude of a point where the user is present, the number of user's steps, the user's speed, the user's step width, and barometric pressure at the point where the user is present.

4. The electronic apparatus according to claim 1,
wherein the processor performs the physical strength evaluation on the basis of an output from at least one of a position sensor, an acceleration sensor, and an atmospheric pressure sensor.

5. The electronic apparatus according to claim 1,
wherein the processor performs the physical condition evaluation on the basis of a temporal change in the user's biological information.

6. The electronic apparatus according to claim 5,
wherein the processor performs the physical condition evaluation on the basis of a temporal change in the user's heart rate.

7. The electronic apparatus according to claim 1,
wherein the processor generates the information representing that the physical activity does not need to be reconsidered when a result of the physical strength evaluation and a result of the physical condition evaluation are both OK.

8. The electronic apparatus according to claim 7,
wherein the processor generates the information representing that the physical activity needs to be changed when, out of a result of the physical strength evaluation and a result of the physical condition evaluation, only the result of the physical condition evaluation is NG.

9. The electronic apparatus according to claim 7,
wherein the processor generates the information representing that the physical activity needs to be temporarily stopped when, out of a result of the physical strength evaluation and a result of the physical condition evaluation, only the result of the physical strength evaluation is NG.

10. The electronic apparatus according to claim 7,
wherein the processor generates the information representing that the physical activity needs to be discontinued when a result of the physical strength evaluation and a result of the physical condition evaluation are both NG.

11. The electronic apparatus according to claim 1,
wherein the processor generates the information on whether or not a plurality of users' physical activity needs to be reconsidered on the basis of the evaluation of the plurality of users' physical strength and the evaluation of the plurality of users' physical condition.

12. The electronic apparatus according to claim 11,
wherein the processor generates the information representing that the physical activity does not need to be reconsidered when results of the evaluation of all the plurality of users' physical strength and results of the evaluation of all the plurality of users' physical condition are both OK.

13. The electronic apparatus according to claim 12,
wherein the processor generates the information representing that the physical activity needs to be changed when the number of users whose results of the physical condition evaluation, out of results of the physical strength evaluation and results of the physical condition evaluation, are only NG is greater than or equal to a predetermined value.

14. The electronic apparatus according to claim 12,
wherein the processor generates the information representing that the physical activity needs to be temporarily stopped when the number of users whose results of the physical strength evaluation, out of results of the physical strength evaluation and results of the physical condition evaluation, are only NG is greater than or equal to a predetermined value.

15. The electronic apparatus according to claim 12,
wherein the processor generates the information representing that the physical activity needs to be discontinued when the number of users whose results of the physical strength evaluation and results of the physical condition evaluation are both NG is greater than or equal to a predetermined value.

16. The electronic apparatus according to claim 1,
wherein the processor provides the user with information.

17. The electronic apparatus according to claim 1,
wherein the electronic apparatus is attachable to a predetermined site of the user.

18. The electronic apparatus according to claim 17,
wherein the predetermined site is an arm or a wrist.

19. A physical activity assistance method comprising generating, on the basis of evaluation of physical strength of a user who is performing physical activity and evaluation of the user's physical condition, information on whether or not the user's physical activity needs to be reconsidered.
